(19) United States
(12) Patent Application Publication
(10) Pub. No.: US 2025/0338057 A1
(43) Pub. Date: Oct. 30, 2025

(54) ACOUSTIC PROCESSING APPARATUS

(71) Applicant: SONY GROUP CORPORATION, Tokyo (JP)

(72) Inventors: Seiya SUGAWARA, Kanagawa (JP); Koji NAGENO, Tokyo (JP)

(73) Assignee: SONY GROUP CORPORATION, Tokyo (JP)

(21) Appl. No.: 18/866,482

(22) PCT Filed: May 29, 2023
(86) PCT No.: PCT/JP2023/019891

§ 371 Date: Nov. 15, 2024

(30) Foreign Application Priority Data
Jun. 6, 2022 (JP) 2022-091282
Feb. 22, 2023 (JP) 2023-025681

(51) Int. Cl. H04R 1/10; A61B 5/00; A61B 5/0205; A61B 5/026; H04R 19/02; H04R 19/04

(52) U.S. Cl. CPC H04R 1/1075; A61B 5/02055; A61B 5/6817; H04R 1/1016; H04R 1/1041; H04R 19/02; H04R 19/04; A61B 5/026; A61B 2562/028; H04R 2201/003

(57) ABSTRACT

Provided is an acoustic processing apparatus in which a microelectromechanical systems (MEMS) device is effectively arranged. The acoustic processing apparatus includes an enclosure, a sound duct extending from the enclosure, and a MEMS device housed in the sound duct.

### CROSS REFERENCE TO RELATED APPLICATIONS

This application claims the benefit of Japanese Priority Patent Application JP 2022-091282 filed on Jun. 6, 2022, and the benefit of Japanese Priority Patent Application JP 2023-025681 filed on Feb. 22, 2023, the entire contents of which are incorporated herein by reference.

### TECHNICAL FIELD

The present disclosure relates to an acoustic processing apparatus.

### BACKGROUND ART

In recent years, a device in which mechanical components, electronic circuits, and electronic components are collectively formed on a substrate including silicon, glass, an organic material, or the like, such as a so-called microelectromechanical systems (MEMS) device, has attracted attention. For example, PTL 1 below discloses a speech chip as a MEMS system chip formed by a semiconductor manufacturing process.

### CITATION LIST

### Patent Literature

PTL 1: JP 2022-13874 A

### SUMMARY

### Technical Problem

PTL 1 proposes a speech chip having a package structure, but does not mention that the MEMS device is effectively disposed in a small acoustic processing apparatus such as an earphone. That is, the technique described in PTL 1 is insufficient from the viewpoint of an effective arrangement of the MEMS device, and there is room for improvement.

An object of the present disclosure is to provide an acoustic processing apparatus that realizes an effective arrangement of a MEMS device.

### Solution to Problem

The present disclosure provides an acoustic processing apparatus, for example, including an enclosure, a sound duct extending from the enclosure, and a MEMS device housed in the sound duct.

### BRIEF DESCRIPTION OF DRAWINGS

FIG. 1 is a diagram for explaining an external configuration example of an earphone device according to a first embodiment.

FIG. 2 is a diagram for explaining a MEMS driver according to the first embodiment.

FIG. 3 is a diagram for explaining a MEMS microphone according to the first embodiment.

FIG. 4 is a diagram for explaining an outline of an internal configuration example of the earphone device according to the first embodiment.

FIG. 5 is a diagram for explaining an internal configuration example of the earphone device according to the first embodiment.

FIG. 6 is a diagram for explaining an acoustic channel in the earphone device according to the first embodiment.

FIG. 7 is a diagram for explaining an acoustic channel in a general earphone device.

FIG. 8 is a diagram for explaining a structure of a human ear.

FIG. 9 is a diagram for explaining an example of a MEMS biosensor according to a second embodiment.

FIG. 10 is a diagram for explaining another example of the MEMS biosensor according to the second embodiment.

FIG. 11 is a diagram for describing a modification.

FIG. 12 is a diagram for describing a modification.

FIG. 13 is a diagram for describing a modification.

### DESCRIPTION OF EMBODIMENTS

Hereinafter, embodiments and the like of the present disclosure will be described with reference to the drawings. Note that the description will be given in the following order.

First Embodiment

Second Embodiment

Modification

The embodiments and the like described below are preferred specific examples of the present disclosure, and the content of the present disclosure is not limited to these embodiments and the like. Note that sizes, positional relationships, and the like of members illustrated in the drawings may be exaggerated for clarity of description, and in order to prevent the illustration from being complicated, only a part of reference numerals may be illustrated, a part of the illustration may be simplified, or hatching of cross sections may be omitted. Furthermore, in the following description, the same names and reference numerals indicate the same or similar members, and redundant description will be appropriately omitted. In addition, directions such as up, down, left, and right directions are defined in consideration of convenience of description, but the present disclosure is not limited to the directions in the description.

### FIRST EMBODIMENT

In the embodiment, an earphone device that can be worn on the user's ear will be described as an example of the acoustic processing apparatus. However, the acoustic processing apparatus according to the present disclosure is not limited to the earphone device, and is applicable to, for example, headphones, hearing aids, sound collectors, and wearable devices other than those described above.

### Exemplary External Configuration of Earphone

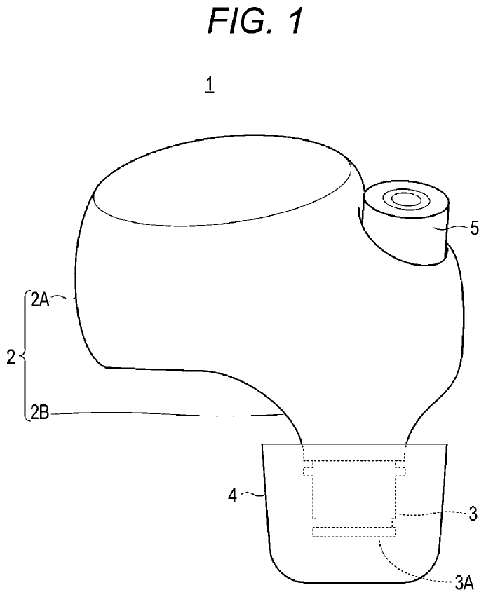

FIG. 1 is a diagram illustrating an external configuration example of an earphone device (earphone device 1) according to an embodiment. The earphone device 1 according to the embodiment reproduces an audio signal wirelessly transmitted from, for example, a portable music player, a smartphone, or the like (not illustrated). Examples of the communication standard of wireless transmission include, but are not limited to, Bluetooth (registered trademark) and a wireless local area network (LAN). Furthermore, the earphone device 1 may be a device that reproduces an audio signal transmitted via a cable.

The earphone device 1 includes, for example, a housing 2 as an enclosure, a substantially cylindrical sound duct 3 extending from the housing 2, an earpiece 4, and a wind detecting microphone 5.

The housing 2 includes a base 2A having a substantially spherical shape or a substantially cylindrical shape, and a protruding portion 2B that is slightly protruding from a predetermined portion (for example, the lower right portion in FIG. 1) of the base 2A. The base 2A and the protruding portion 2B are, for example, integrally molded, but may be configured as separate members and may have a configuration in which they are attached. The housing 2 includes, for example, acrylonitrile butadiene styrene (ABS) resin. In the base 2A and the protruding portion 2B, an internal space communicating with each other is formed, and for example, a battery serving as a power source of earphone device 1, a circuit for wireless communication, a circuit for sound processing, and the like are housed in the internal space of base 2A (to be described later in detail).

A hole is formed in the end surface of the distal end of the protruding portion 2B, and the sound duct 3 extends from the hole toward the outside of the housing 2. The sound duct 3 includes, for example, ABS resin, and is molded integrally with the housing 2. The sound duct 3 may be configured separately from the housing 2 and attached to the housing 2. The sound duct 3 has a cylindrical shape with an internal space and has a sound duct end 3A with an opening. When the earphone device 1 is worn on the user's ear, the sound is led out from the sound duct end 3A into the user's ear.

Although details will be described later, a MEMS device is housed in the sound duct 3. The MEMS device includes at least one of a MEMS driver, a MEMS microphone, or a MEMS biosensor. The present embodiment is an example in which the MEMS device includes a MEMS driver and a MEMS microphone. For example, a diaphragm in the MEMS driver vibrates on the basis of a wirelessly transmitted audio signal. When the diaphragm vibrates, a sound corresponding to the audio signal is generated, and the generated sound is reproduced from the sound duct end 3A into the user's ear. The earpiece 4 includes silicone rubber, urethane-based resin, acrylic resin, or the like, and is an elastically deformable attachment member. The earpiece 4 has a hole therein, and as illustrated in FIG. 1, the earpiece 4 is attached to the outside of the sound duct 3 by inserting the sound duct 3 into the hole. Since the earpiece 4 can be elastically deformed, the diameter thereof slightly increases when the earpiece 4 is inserted into the sound duct 3, and the earpiece 4 can be smoothly inserted into the sound duct 3. Note that the earpiece 4 may cover not only the sound duct 3 but also the outside of a part of the protruding portion 2B. The earpiece 4 has an opening at the distal end, and is configured not to inhibit sound emitted from the sound duct end 3A from being directed into the ear. The earpiece 4 may have a mesh-like configuration without an opening. In addition, in a case where the earphone device 1 is worn on the user's ear, the earpiece 4 is elastically deformed to come into close contact with the ear canal of the user's ear. As a result, it is possible to prevent the reproduced sound from leaking from the sound duct 3 to the outside. Furthermore, by using the earpiece 4, it is possible to prevent discomfort and pain from being given to the user by the sound duct 3 being in direct contact with the ear canal.

The wind detecting microphone 5 is a microphone that detects wind around earphone device 1. When wind is detected by the wind detecting microphone 5, the microphone used for feed-forward noise cancellation is controlled to be turned off, and wind noise is automatically reduced. Note that the wind detecting microphone 5 may not be provided.

### Example of MEMS Device

Next, a MEMS driver (MEMS driver 10) and a MEMS microphone (MEMS microphone 20), which are examples of the MEMS device according to the present embodiment, will be described. Note that, in the present specification, the MEMS device is a device formed by a microfabrication technology (MEMS process) to which a semi-conductor element manufacturing process is applied. The MEMS devices herein may also include mechanical components that are not formed in a MEMS process as part of the configuration. However, from the viewpoint of further downsizing the entire MEMS device, it is preferable that the entire MEMS device is automatically formed by the MEMS process.

An example of the MEMS process will be described. The MEMS process includes, for example, the following steps.
S1 Film forming step: a thin film of a mask material is formed on a substrate of silicon or the like.
S2 Photolithography step: a resist (photosensitive resin) is applied or attached onto the thin film, and a pattern is formed by light irradiation through a photomask.
S3 Etching step: unnecessary portions of the thin film, the silicon substrate, or the like are scraped off using a gas or a chemical solution.
S4 Bonding step: a plurality of silicon substrates are bonded.
S5 Completion step: The MEMS device is completed by performing a cutting (dicing) process and a packaging process.
   Note that the MEMS process described above is an example of one MEMS process, and some steps may be omitted or other steps may be added.

A general dynamic type driver is formed by assembling mechanical components. Such a dynamic-type driver has limitations in miniaturization and assembly processes of individual components, and as a result, there is a limitation in miniaturization. Since the MEMS driver can be produced only by the MEMS process, it is possible to obtain an advantage that downsizing (miniaturization) can be achieved. In addition, it is possible to automatically perform mass production, and thus, it is possible to obtain superiority in price. These advantages can be applied not only to the MEMS driver but also to the MEMS microphone and the MEMS biosensor.

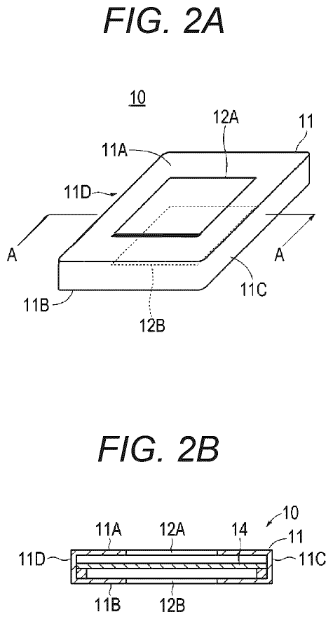

FIG. 2A illustrates an external appearance example of the MEMS driver 10 according to the present embodiment, and FIG. 2B illustrates a cross section of the MEMS driver 10 taken along line A-A in FIG. 2A. As illustrated in FIG. 2A, the MEMS driver 10 according to the present embodiment includes an enclosure 11 having a schematically rectangular parallelepiped shape (chip shape). The enclosure 11 has an upper surface 11A and a bottom surface 11B opposite to the upper surface 11A. The upper surface 11A is an upper main surface (a surface having a relatively large area with respect to other surfaces) in FIG. 2A, and the bottom surface 11B is a lower main surface in FIG. 2A. A hole 12A (an example of a first hole) functioning as a sound hole is formed in the upper surface 11A. A hole 12B functioning as a sound hole is formed in the bottom surface 11B. The hole 12A and the hole 12B are, for example, holes having a rectangular shape.

As illustrated in FIG. 2B, a diaphragm 14 is provided in the enclosure 11. The diaphragm 14 is supported by a support member or the like so as to bridge over the left and right side surfaces 11C and 11D in FIGS. 2A and 2B. As described above, each configuration of the MEMS driver 10 is formed by the MEMS process. The diaphragm 14 vibrates on the basis of the audio signal supplied to the MEMS driver 10, whereby a sound corresponding to the audio signal is generated. The generated sound is emitted from the hole 12A. In addition, a sound generated by vibration of the diaphragm 14 and having a phase opposite to that of the sound emitted from the hole 12A is emitted from the hole 12B.

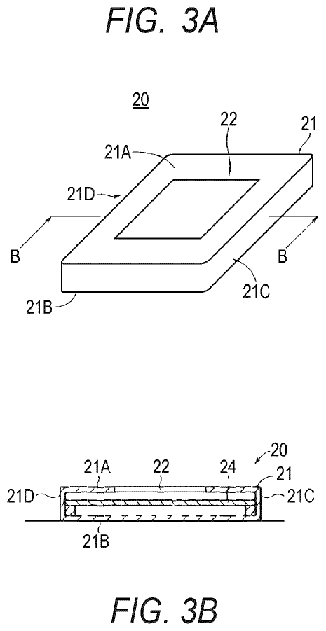

FIG. 3A illustrates an external appearance example of the MEMS microphone 20 according to the present embodiment, and FIG. 3B illustrates a cross section of the MEMS microphone 20 taken along line B-B in FIG. 3A. As illustrated in FIG. 3A, the MEMS microphone 20 according to the present exemplary embodiment includes an enclosure 21 having a schematically rectangular parallelepiped shape (chip shape). For example, the hole 22 (an example of a second hole) functioning as a sound hole is formed in the upper surface 21A of the enclosure 21. The hole 22 is, for example, a hole having a rectangular shape. As illustrated in FIG. 3B, a diaphragm 24 is provided in the enclosure 21. The diaphragm 24 is supported by a support member or the like so as to bridge over the left and right side surfaces 21C and 21D in FIGS. 3A and 3B. As described above, each configuration of the MEMS microphone 20 is formed by the MEMS process.

The MEMS microphone 20 is a microphone that collects reproduced sound reproduced from the MEMS driver 10 described above. That is, reproduced sound emitted from the hole 12A of the MEMS driver 10 is taken into the enclosure 21 through the hole 22 of the MEMS microphone 20. When the diaphragm 24 vibrates due to sound taken into the enclosure 21, reproduced sound from the MEMS driver 10 is collected and detected. Note that the sound collected by the MEMS microphone 20 may include not only the sound reproduced from the MEMS driver 10 but also noise.

The MEMS microphone 20 is used as, for example, a feedback noise canceling microphone for performing noise cancellation by a feedback method. A signal having a phase opposite to that of a sound signal collected by the MEMS microphone 20 and possibly including noise is generated as a noise cancellation signal. By performing the known noise cancellation process using the noise cancellation signal, noise that can be included in the sound reproduced from the MEMS driver 10 is removed or reduced.

As the MEMS driver 10 and the MEMS microphone 20, a known device other than the above-described configuration can also be applied.

### Internal Configuration Example of Earphone Device

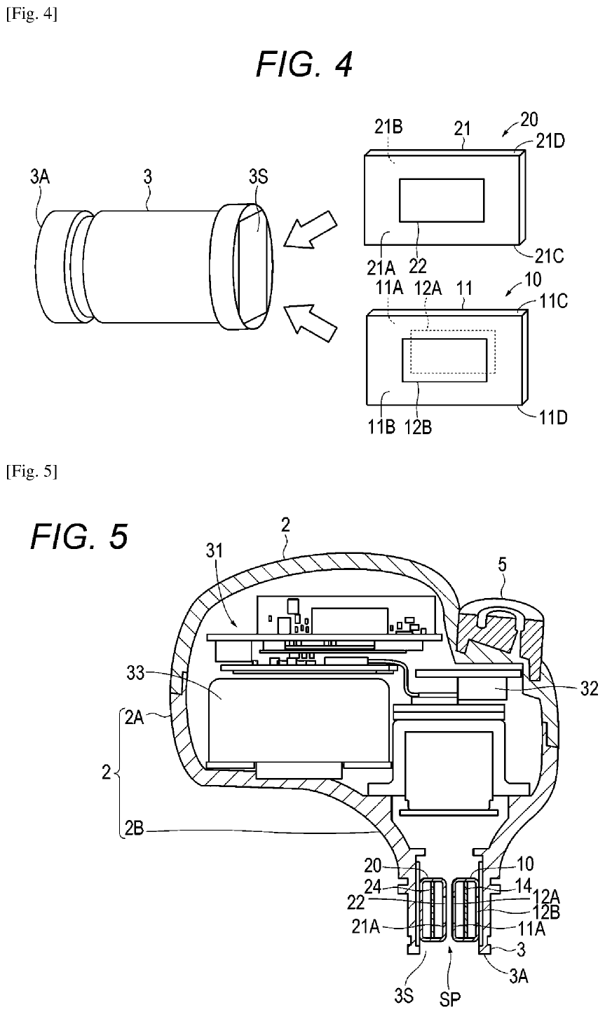

Next, an internal configuration example of the earphone device 1 according to the present embodiment will be described with reference to FIGS. 4 and 5. As illustrated in FIG. 4, the sound duct 3 according to the present embodiment has an internal space 3S. One side of the internal spaces 3S communicates with the internal space of the housing 2, and the other side of the internal space 3S is a sound duct end 3A. The MEMS driver 10 and the MEMS microphone 20 are housed in the internal space 3S. For example, the MEMS driver 10 and the MEMS microphone 20 are housed in the internal space 3S such that the upper surface 11A of the MEMS driver 10 and the upper surface 21A of the MEMS microphone 20 face each other and a gap (gap SP to be described later) is formed between the upper surface 11A and the upper surface 21A. As described above, since the MEMS driver 10 and the MEMS microphone 20 can be downsized, both can be housed even in the sound duct 3 (for example, a sound duct having a diameter of about several millimeters) having a relatively small diameter.

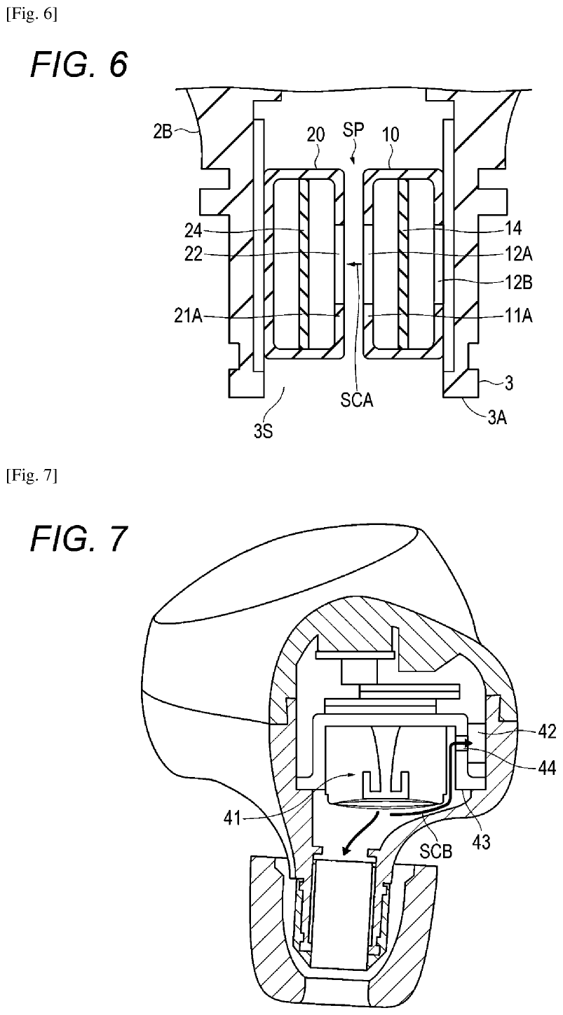

FIG. 5 is a cross-sectional view illustrating a detailed internal configuration example of the earphone device 1 according to the present embodiment. Note that the cross-sectional views of FIGS. 5 and 6 are cross-sectional views of a state where the earpiece 4 is not attached. In the base 2A, for example, a circuit unit 31, a feedforward noise canceling microphone 32 for performing feedforward noise cancellation, and a battery 33 are housed.

The circuit unit 31 is a generic term for a communication circuit that receives an audio signal from an external device such as a smartphone or a portable audio player, an audio processing circuit that performs known audio processing, an amplifier that amplifies an audio signal, a circuit that performs a noise cancellation process, and the like. Each unit of earphone device 1 such as the circuit unit 31 operates on the basis of power supplied from the battery 33. A circuit that performs noise cancellation in the circuit unit 31 is connected to the MEMS microphone 20 that is a feedback noise canceling microphone and the feedforward noise canceling microphone 32. The circuit unit 31 is also connected to the MEMS driver 10, and is configured such that the audio signal received from the external device by the circuit unit 31 is appropriately amplified and then supplied to the MEMS driver 10. Note that illustration of these connection patterns is simplified or omitted as appropriate.

As described above, the MEMS driver 10 and the MEMS microphone 20 are housed in the internal space 3S of the sound duct 3. The MEMS driver 10 and the MEMS microphone 20 may be attached to the inner surface of the sound duct 3 by adhesion or the like, may be attached by an appropriate support member, or may be fitted. In a state where the MEMS driver 10 and the MEMS microphone 20 are housed in the internal space 3S, a gap SP is formed between the upper surface 11A of the MEMS driver 10 and the upper surface 21A of the MEMS microphone 20. The reproduction sound reproduced from the MEMS driver 10 reaches the eardrum of the user via the gap SP and the open end of the sound duct 3. The reproduction sound reproduced from the MEMS driver 10 reaches the MEMS microphone 20 through the gap SP and is detected. Note that since the hole 12B of the MEMS driver 10 faces the inner surface of the sound duct 3, propagation of a sound having a phase opposite to that of the reproduction sound of the MEMS driver 10 to the eardrum side is suppressed.

Since the MEMS driver 10 and the MEMS microphone 20 can be housed in the sound duct 3, the earphone device 1 can be downsized. For example, in the related art, in order to house a dynamic-type driver in the housing 2, the housing 2 needs to have a certain volume or more. However, since the configuration related to the driver can be housed in the sound duct 3, the housing 2 can be downsized, and the entire earphone device 1 can be downsized. That is, effective arrangement of the MEMS device included in the acoustic processing apparatus can be realized. In addition, since the earphone device 1 can be downsized, the internal volume of the ear canal, which is an acoustic load, can be minimized. That is, since the earphone device 1 can be inserted relatively deep into the ear, the volume of air (acoustic load) from the diaphragm 14 to the eardrum through the ear canal can be reduced. As a result, since the volume of air can be reduced with respect to a constant amplitude of the diaphragm 14, the generated AC atmospheric pressure can be increased, and the earphone device 1 can be a highly sensitive transducer. Furthermore, since the MEMS driver 10 is a miniaturized device, sound can be reproduced by vibrating a relatively small amount of air in the device, and sensitivity can be improved.

Further, according to the configuration of the present exemplary embodiment, MEMS driver 10 and MEMS microphone 20 that is a feedback noise canceling microphone can be disposed close to each other. That is, as indicated by an arrow in the partially enlarged view of FIG. 6, the reproduced sound reproduced by the MEMS driver 10 can be collected by the MEMS microphone 20 disposed near the MEMS driver 10, and an acoustic channel SCA, which is the propagation distance of the reproduced sound, can be shortened.

FIG. 7 illustrates an internal configuration example of a general earphone device. The earphone device shown in FIG. 7 has a dynamic-type driver unit 41 in a housing. A feedback noise canceling microphone 42 is disposed at a position close to the driver unit 41. A hole 44 connected to an opening of the feedback noise canceling microphone 42 is formed in a wall 43 in which the driver unit 41 is housed. As schematically indicated by an arrow in FIG. 7, the reproduction sound reproduced by the driver unit 41 reaches the feedback noise canceling microphone 42 via the hole 44 of the wall 43. In the case of a general earphone device, the length of an acoustic channel SCB until the reproduction sound reproduced from the driver unit 41 reaches the feedback noise canceling microphone 42 increases. For example, the length of the acoustic channel SCB becomes about 10 mm.

When the length of the acoustic channel SCB becomes about 10 mm, for example, the phase is rotated (inverted) by about 60 degrees before the reproduction sound of 5 kHz reaches the feedback noise canceling microphone 42. In a case where the rotation of the phase is about 60 degrees, the noise cancellation effect of the feedback method becomes extremely small.

In order to prevent such rotation of the phase of the reproduction sound and obtain the effect of noise cancellation, it is desirable to reduce the length of the acoustic channel of the reproduction sound as much as possible. In the configuration of the present embodiment, since the MEMS driver 10 and the MEMS microphone 20 can be downsized, the MEMS driver 10 and the MEMS microphone 20 can be arranged close to each other in the sound duct 3. That is, the length of the acoustic channel of the reproduction sound reproduced from the MEMS driver 10 can be reduced. For example, in the configuration according to the present embodiment, the length of the acoustic channel SCA until the reproduction sound reproduced from the MEMS driver 10 reaches the MEMS microphone 20 can be set to 3 mm or less. In a case where the length of the acoustic channel SCA is 3 mm or less, for example, the rotation of the phase of the reproduction sound of 5 kHz is 20 degrees or less, and the effect of noise cancellation by the feedback method can be sufficiently obtained. The length of the acoustic channel SCA can be defined by, for example, the shortest distance in the sound propagation space from the open end surface of the hole 12A of the MEMS driver 10 to the diaphragm 24 of the MEMS microphone 20.

### SECOND EMBODIMENT

Next, a second embodiment will be described. Note that, in the description of the second embodiment, the same or similar configurations in the above description are denoted by the same reference numerals, and redundant description is appropriately omitted. In addition, the matters described in the first embodiment can be applied to the second embodiment unless otherwise specified.

In the second embodiment, the MEMS device housed in the sound duct 3 includes a MEMS driver 10, a MEMS microphone 20, and a MEMS biosensor. The MEMS biosensor is a biosensor formed by the above-described MEMS process. Examples of the MEMS biosensor include a blood flow sensor, a heart rate/pulse sensor, an electroencephalography (EEG) sensor, and a body temperature sensor. The MEMS biosensor may be a sensor that acquires biological data other than the above-described biological data.

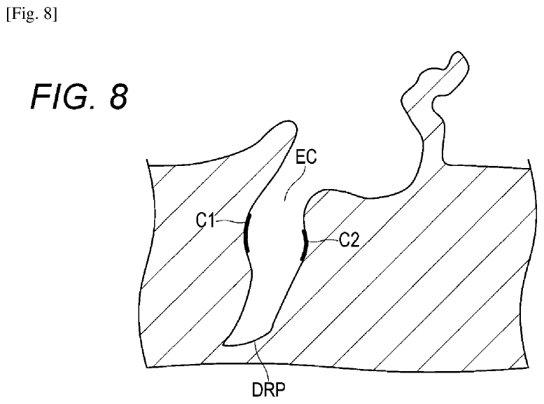

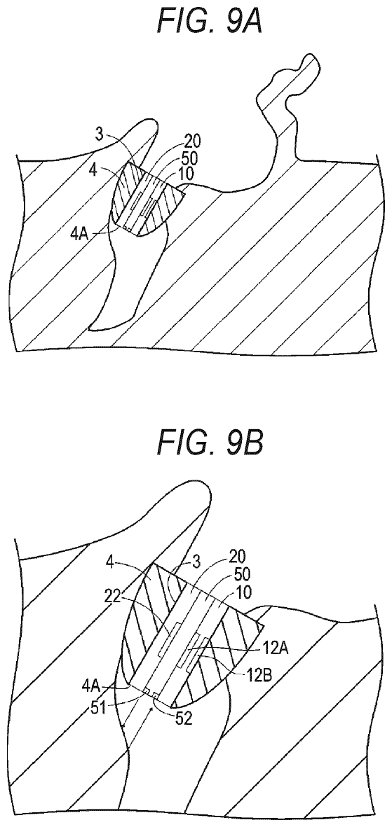

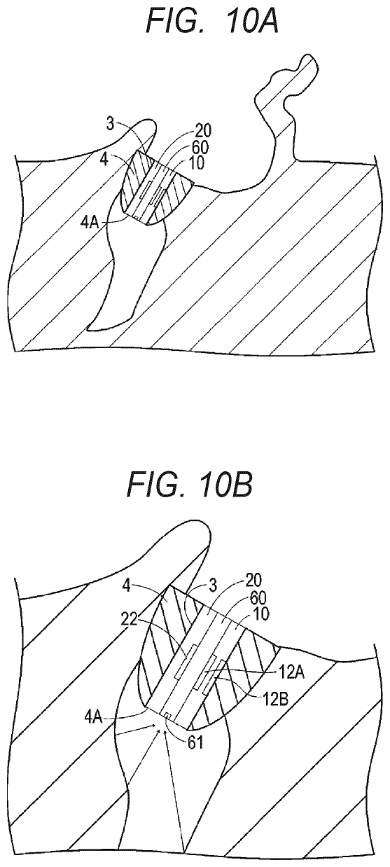

FIG. 8 illustrates a structure of a general human ear canal EC. The ear canal EC comes to the end of the first curve C1 at a depth of about 10 mm from the entrance, and further reaches the eardrum DRP through the second curve C2 about 10 mm ahead. The earpiece 4 is inserted into the first curve C1 from the entrance of the ear canal EC, and an opening 4A (see FIGS. 9 and 10) at the distal end of the earpiece 4 faces the ear wall near the first curve C1. Many blood vessels pass through the subcutaneous tissue in the ear wall of a human, which is a suitable site for observing the blood flow of the human body.

Examples of a method of measuring blood flow include a method of observing hemoglobin in blood. In such a method, the blood flow sensor includes a light source that irradiates a blood flow portion with infrared rays and a light receiving element that receives reflected light. In principle, since light of a specific wavelength is absorbed by hemoglobin in the blood stream, it is possible to confirm a change in the amount of hemoglobin (contraction of blood vessels, that is, pulse) by observing the wavelength of the reflected light.

As illustrated in FIG. 9A, the MEMS driver 10, the MEMS microphone 20, and the blood flow sensor 50, which is an example of the MEMS biosensor, are housed in the sound duct 3 to which the earpiece 4 is attached. As an example, the MEMS driver 10 and the MEMS microphone 20 are arranged so as to be in contact with the inner surface of the sound duct 3, and the blood flow sensor 50 is arranged therebetween. In FIGS. 9A and 9B, illustration of the earphone device is simplified. Further, a propagation path (space) of the reproduction sound toward the eardrum and a propagation path toward the MEMS microphone 20 are omitted as appropriate. Furthermore, the arrangement position of the blood flow sensor 50 may be a position other than between the MEMS driver 10 and the MEMS microphone 20, for example, a surface facing the inside of the ear canal EC of the MEMS driver 10 or the MEMS microphone 20.

As illustrated in FIG. 9B, the blood flow sensor 50 includes a light source 51 and a light receiving element 52.

The light source 51 and the light receiving element 52 are arranged to face the ear wall near the first curve C1 through the opening 4A at the distal end of the earpiece 4. The light source 51 and the light receiving element 52 are connected to the circuit unit 31 by a wiring pattern (not illustrated). Light emission of the light source 51 is controlled by an integrated circuit (IC) of the circuit unit 31. In addition, a signal received by the light receiving element 52 and converted into an electric signal is supplied to the circuit unit 31, and a known process for measuring blood flow is performed by the IC of the circuit unit 31. Note that an IC that controls the light source 51 and processes the light receiving signal of the light receiving element 52 may be integrated with the blood flow sensor 50 by the MEMS process.

As illustrated in FIG. 9B, the infrared light emitted from the light source 51 is applied to the ear wall near the first curve C1, and the reflected light is received by the light receiving element 52. In FIG. 9B, the infrared light and the reflected light are schematically indicated by arrows. Then, the blood flow of the user of the earphone device is measured by the above-described principle.

As described above, since the blood flow sensor 50 formed by the MEMS process can be downsized, the blood flow sensor 50 can be housed in the sound duct 3. Therefore, it is possible to effectively measure the blood flow of the user of the earphone device without increasing the size of the earphone device.

As described above, the earphone device may be a hearing aid. In this case, it is possible to continue to observe blood flow while compensating for hearing with a hearing aid that is considered to be typically worn by an elderly person who feels impaired hearing. The obtained data regarding the blood flow may be transmitted from the hearing aid to a smartphone of an elderly person, a server for monitoring a health condition, or the like. By transmitting data regarding blood flow to an external device, it is possible to construct a health condition monitoring system, a system that reports an abnormality to a family member of an elderly person or a caregiver in a case where an abnormality is recognized in blood flow, and the like.

The MEMS biosensor may be a body temperature sensor instead of the blood flow sensor 50.

Since the inside of the auditory canal is closer to the internal body temperature than the body surface temperature and is constant without being affected by the external temperature, a non-contact type thermometer is in practical use. In principle, infrared rays corresponding to the body temperature are emitted from the interior of the auditory canal, and the body temperature can be measured by detecting the infrared rays with the infrared light receiving element.

As illustrated in FIG. 10A, a MEMS driver 10, a MEMS microphone 20, and a body temperature sensor 60, which is an example of a MEMS biosensor, are housed in the sound duct 3 to which the earpiece 4 is attached. As an example, the MEMS driver 10 and the MEMS microphone 20 are arranged so as to be in contact with the inner surface of the sound duct 3, and the body temperature sensor 60 is arranged therebetween. In FIGS. 10A and 10B, illustration of the earphone device is simplified. Further, a propagation path (space) of the reproduction sound toward the eardrum and a propagation path toward the MEMS microphone 20 are omitted as appropriate. Furthermore, the arrangement position of the body temperature sensor 60 may be a position other than between the MEMS driver 10 and the MEMS microphone 20, for example, a surface facing the inside of the ear canal EC of the MEMS driver 10 or the MEMS microphone 20.

As illustrated in FIG. 10B, the body temperature sensor 60 includes an infrared light receiving element 61. The infrared light receiving element 61 is disposed so as to face the ear wall near first curve C1 through the opening 4A at the distal end of earpiece 4. Infrared light (schematically indicated by an arrow in FIG. 10B) emitted from the ear wall is received by the infrared light receiving element 61. The infrared light receiving element 61 is connected to the circuit unit 31 by a wiring pattern (not illustrated). A signal received by the infrared light receiving element 61 and converted into an electric signal is supplied to the circuit unit 31. Then, known processing for measuring the body temperature is performed by the IC of the circuit unit 31. Note that an IC that processes the light receiving signal of the infrared light receiving element 61 may be integrated with the body temperature sensor 60 by the MEMS process.

In this manner, since the body temperature sensor 60 formed by the MEMS process can be downsized, the body temperature sensor 60 can be housed in the sound duct 3. This makes it possible to effectively measure the body temperature of the user of the earphone device without increasing the size of the earphone device.

It is considered that not only at the time of listening to music by the earphone device but also a life style in which the earphone device is typically worn and voice information is received via a conversation with the surroundings or a network such as the Internet becomes widespread in the future. In this case, since the body temperature, which is the basic information of health, can be continuously measured, the user can receive not only health management in the life cycle of the user of the earphone device but also provision of various information services linked to the content of the activity. For example, when the body temperature is high, the user can receive, via the earphone device, services such as provision of medical information by voice and provision of music that calms the user.

Note that, in the above-described example, the blood flow sensor 50 or the body temperature sensor 60 has been described as an example of the MEMS biosensor. However, both of the sensors may be included in the MEMS biosensor, or other biosensor (for example, a blood pressure sensor, a heart rate/pulse sensor, or an EEG sensor) may be used instead of the blood flow sensor 50 and the body temperature sensor 60, or a combination thereof may be used. Since the MEMS biosensor can be downsized, a plurality of MEMS biosensors can be housed in the sound duct 3.

### MODIFICATION

Although the embodiment of the present disclosure has been specifically described above, the content of the present disclosure is not limited to the above-described embodiment, and various modifications based on the technical idea of the present disclosure are possible.

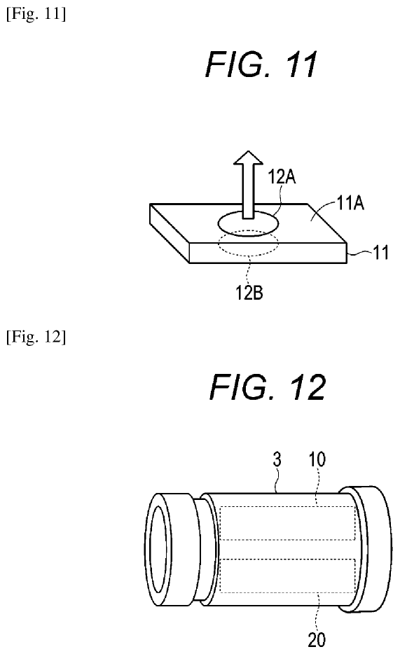

The shape of the MEMS device may be a shape other than that described in the embodiment. For example, the enclosure 11 of the MEMS driver 10 may have a cylindrical shape. In addition, as illustrated in FIG. 11, the shape of the hole 12A and the hole 12B is not limited to a rectangular shape, and may be a circular shape, or may be another shape, for example, an elliptical shape, a polygonal shape, or the like. The same applies to the shape of the hole 22.

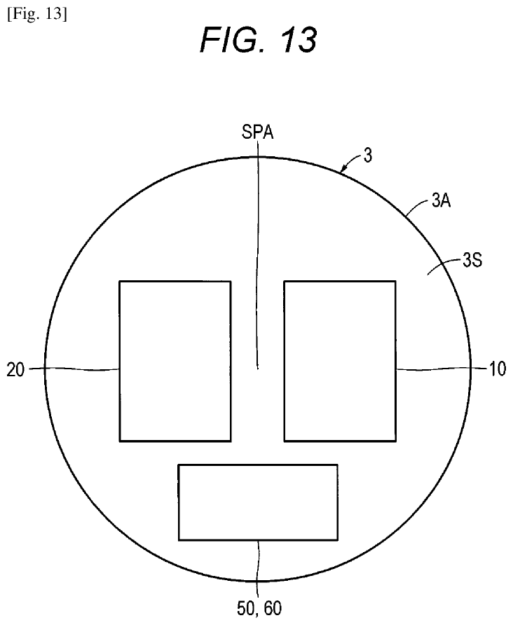

In the above-described embodiment, the MEMS driver and the MEMS microphone are described as separate MEMS devices, but the MEMS device may be a MEMS device in which the MEMS driver and the MEMS microphone are integrally configured by one MEMS process. In addition, the MEMS device may be a MEMS device in which the MEMS driver, the MEMS microphone, and the MEMS biosensor are integrally configured by one MEMS process. Furthermore, as illustrated in FIG. 12, the sound duct 3, the MEMS driver 10, and the MEMS microphone 20 may be a MEMS device integrally configured by one MEMS process. For example, the MEMS driver 10 and the MEMS microphone 20 may be formed on the inner surface of the sound duct 3 obtained by shape processing of a silicon substrate or the like by the MEMS process. Furthermore, the MEMS device may have a configuration in which the MEMS biosensor is further integrated with the configuration illustrated in FIG. 12. The positions where the MEMS driver, the MEMS microphone, and the MEMS biosensor are disposed are not limited to the examples described in the embodiment. FIG. 13 is a diagram for describing an arrangement example according to the present modification of the MEMS driver 10, the MEMS microphone 20, and the MEMS biosensor (for example, the blood flow sensor 50 and the body temperature sensor 60 described above). Note that FIG. 13 illustrates an arrangement example (top view) of the MEMS driver 10 and the like when the internal space 3S of the sound duct 3 is viewed from the sound duct end 3A side.

As illustrated in FIG. 13, the MEMS driver 10 and the MEMS microphone 20 are disposed so as to form a gap SPA therebetween. The MEMS biosensor 50, 60 may be disposed at a position other than the gap SPA. For example, the MEMS biosensor 50, 60 may be disposed at a position below the MEMS driver 10 and the MEMS microphone 20 in the internal space 3S. The MEMS biosensor 50, 60 may be disposed at a position above the MEMS driver 10 and the MEMS microphone 20 in the internal space 3S. Further, different MEMS biosensors 50, 60 may be disposed above and below the MEMS driver 10 and the MEMS microphone 20.

Although the combination of the MEMS devices is preferably the combination described in the embodiment, for example, the MEMS device housed in the sound duct may be any one of the MEMS driver, the MEMS microphone, and the MEMS biosensor.

The material of the housing 2 and the sound duct 3 is not limited to the ABS resin, and for example, various other resins such as polypropylene and polystyrene may be used. Further, a flexible resin such as an elastomer resin may be used as the material of the protruding portion 2B. In this case, since the portion of the protruding portion 2B has flexibility, the sound duct 3 can be bent. As a result, when the earphone device is worn on the user's ear, the earphone device can be worn by bending the sound duct 3 and the earpiece 4 in a direction with a better wearing feeling.

The acoustic processing apparatus according to the present disclosure can also be configured as a hearing aid or a sound collector. In the case of use as a hearing aid, a phenomenon occurs in which the sound emitted by the user can be heard loudly, or the sound transmitted by the user's mastication sound or stepping on the foot stays in the hearing aid. These phenomena are called occlusion.

As described in the first embodiment and the like, the performance of noise cancellation can be improved by accommodating the MEMS driver and the MEMS microphone in the sound duct. By improving the noise canceling performance, the above-described occlusion can be suppressed, and a hearing aid having excellent performance can be provided.

The configurations, methods, steps, shapes, materials, numerical values, and the like described in the above-described embodiments are merely examples, and different configurations, methods, steps, shapes, materials, numerical values, and the like may be used as necessary. The above-described embodiments and modifications can be appropriately combined.

The present disclosure can also adopt the following configurations.

(1) An acoustic processing apparatus including:
an enclosure;
a sound duct extending from the enclosure; and
a MEMS device housed within the sound duct.

(2) The acoustic processing apparatus according to (1), in which the MEMS device includes at least one of a MEMS driver, a MEMS microphone, or a MEMS biosensor.

(3) The acoustic processing apparatus according to (2), in which the MEMS device includes the MEMS driver and the MEMS microphone.

(4) The acoustic processing apparatus according to (3), in which
the MEMS driver includes a first hole,
the MEMS microphone includes a second hole, and
a distance between the first hole and the second hole is 3 mm or less.

(5) The acoustic processing apparatus according to (3) or (4), in which the MEMS driver and the MEMS microphone are integrally configured.

(6) The acoustic processing apparatus according to any one of (3) to (5), in which the MEMS microphone is a microphone that collects a sound reproduced from the MEMS driver.

(7) The acoustic processing apparatus according to (2), in which the MEMS device includes the MEMS driver, the MEMS microphone, and the MEMS biosensor.

(8) The acoustic processing apparatus according to (7), in which the MEMS driver, the MEMS microphone, and the MEMS biosensor are integrally configured.

(9) The acoustic processing apparatus according to any one of (2) to (8), in which
the MEMS device includes the MEMS biosensor, and
the MEMS biosensor includes at least one of a blood flow sensor or a body temperature sensor.

(10) The acoustic processing apparatus according to any one of (1) to (9), in which the sound duct and the MEMS device are configured as an integrated MEMS device.

(11) The acoustic processing apparatus according to any one of (1) to (10), further including an attachment member that is attached to an outside of the sound duct and is elastically deformable.

(12) The acoustic processing apparatus according to any one of (1) to (11), in which the sound duct has a substantially cylindrical shape.

(13) The acoustic processing apparatus according to any one of (1) to (11), wherein the MEMS driver includes a third hole and a first diaphragm.

(14) The acoustic processing apparatus according to (13), wherein the first diaphragm is disposed between the at least first hole and the third hole.

(15) The acoustic processing apparatus according to (13) or (14), wherein the MEMS microphone includes a second diaphragm and the second and third holes are provided between the first and second diaphragms.

(16) The acoustic processing apparatus according to (9), wherein the MEMS biosensor is provided above each of the MEMS driver and the MEMS microphone in a plan view.

(17) The acoustic processing apparatus according to (9), wherein the MEMS biosensor is provided below each of the MEMS driver and the MEMS microphone in a plan view.

(18) The acoustic processing apparatus according to (9) wherein the MEMS biosensor includes a blood flow sensor and a body temperature sensor.

(19) The acoustic processing apparatus according to (18) wherein one of the blood flow sensor and a body temperature sensor is provided above each of the MEMS driver and the MEMS microphone and another of the blood sensor and the body temperature sensor is provided below each of the MEMS driver and the MEMS microphone.

(20) An acoustic system, comprising:
a sound source creating a sound wave; and
an acoustic processing apparatus receiving the sound wave, the acoustic processing apparatus, comprising:
an enclosure;
a sound duct extending from the enclosure; and
a microelectromechanical systems (MEMS) device housed within the sound duct.

It should be understood by those skilled in the art that various modifications, combinations, sub-combinations and alterations may occur depending on design requirements and other factors insofar as they are within the scope of the appended claims or the equivalents thereof.

### REFERENCE SIGNS LIST

1 Earphone device
2 Enclosure
3 Sound duct
4 Earpiece
10 MEMS driver
20 MEMS microphone
50 Blood flow sensor
60 Body temperature sensor

What is claimed is:

1. An acoustic processing apparatus, comprising:
an enclosure;
a sound duct extending from the enclosure; and
a microelectromechanical systems (MEMS) device housed within the sound duct.

2. The acoustic processing apparatus according to claim 1, wherein
the MEMS device includes at least one of a MEMS driver, a MEMS microphone, or a MEMS biosensor.

3. The acoustic processing apparatus according to claim 2, wherein
the MEMS device includes the MEMS driver and the MEMS microphone.

4. The acoustic processing apparatus according to claim 3, wherein
the MEMS driver includes at least a first hole,
the MEMS microphone includes at least a second hole, and
a distance between the at least first hole and the at least second hole is 3 mm or less.

5. The acoustic processing apparatus according to claim 3, wherein
the MEMS driver and the MEMS microphone are integrally configured.

6. The acoustic processing apparatus according to claim 3, wherein
the MEMS microphone is a microphone that collects a sound reproduced from the MEMS driver.

7. The acoustic processing apparatus according to claim 2, wherein
the MEMS device includes the MEMS driver, the MEMS microphone, and the MEMS biosensor.

8. The acoustic processing apparatus according to claim 7, wherein
the MEMS driver, the MEMS microphone, and the MEMS biosensor are integrally configured.

9. The acoustic processing apparatus according to claim 2, wherein
the MEMS device includes the MEMS biosensor, and
the MEMS biosensor includes at least one of a blood flow sensor, a heart rate sensor, an electroencephalography sensor, a blood pressure sensor, a blood glucose level sensor or a body temperature sensor.

10. The acoustic processing apparatus according to claim 1, wherein
the sound duct and the MEMS device are configured as an integrated MEMS device.

11. The acoustic processing apparatus according to claim 1, further comprising
an attachment member that is attached to an outside of the sound duct and is elastically deformable.

12. The acoustic processing apparatus according to claim 1, wherein
the sound duct has a substantially cylindrical shape.

13. The acoustic processing apparatus according to claim 3, wherein the MEMS driver includes a third hole and a first diaphragm.

14. The acoustic processing apparatus according to claim 13, wherein the first diaphragm is disposed between the at least first hole and the third hole.

15. The acoustic processing apparatus according to claim 13, wherein the MEMS microphone includes a second diaphragm and the second and third holes are provided between the first and second diaphragms.

16. The acoustic processing apparatus according to claim 9, wherein the MEMS biosensor is provided above each of the MEMS driver and the MEMS microphone in a plan view.

17. The acoustic processing apparatus according to claim 9, wherein the MEMS biosensor is provided below each of the MEMS driver and the MEMS microphone in a plan view.

18. The acoustic processing apparatus according to claim 9 wherein the MEMS biosensor includes a blood flow sensor and a body temperature sensor.

19. The acoustic processing apparatus according to claim 18 wherein one of the blood flow sensor and a body temperature sensor is provided above each of the MEMS driver and the MEMS microphone and another of the blood sensor and the body temperature sensor is provided below each of the MEMS driver and the MEMS microphone.

20. An acoustic system, comprising:
a sound source creating a sound wave; and
an acoustic processing apparatus receiving the sound wave, the acoustic processing apparatus, comprising:
an enclosure;
a sound duct extending from the enclosure; and
a microelectromechanical systems (MEMS) device housed within the sound duct.
